(19) United States
(12) Patent Application Publication
(10) Pub. No.: US 2018/0059080 A1
(43) Pub. Date: Mar. 1, 2018

(54) HIGHLY SENSITIVE CARBON-NANOMATERIAL-BASED GAS SENSOR FOR USE IN HIGH-HUMIDITY ENVIRONMENT

(71) Applicant: INDUSTRY-ACADEMIC COOPERATION FOUNDATION YONSEI UNIVERSITY, Seoul (KR)

(72) Inventors: Seong Chan JUN, Seoul (KR); Youngmo JUNG, Goyang-si (KR); Seok LEE, Seoul (KR); Taikjin LEE, Seoul (KR); Chulki KIM, Seoul (KR); Chong Yun KANG, Seoul (KR); Sang Kyung KIM, Seoul (KR)

(21) Appl. No.: 15/693,691

(22) Filed: Sep. 1, 2017

(30) Foreign Application Priority Data
Sep. 1, 2016 (KR) 10-2016-0112309

(51) Int. Cl. G01N 33/00

(52) U.S. Cl. CPC G01N 33/0013

(57) ABSTRACT

A highly sensitive carbon-nanomaterial-based gas sensor for use in high-humidity environments and a method of improving the sensitivity thereof, the gas sensor being configured such that a functional group for binding to a water molecule is formed on the surface of a first detector composed of a carbon nanomaterial, whereby a hydronium ion (H₃O⁺) is produced and thus an additional ion conduction path is formed, thereby obtaining an additional reaction path in high-humidity environments, ultimately improving the sensitivity and detection threshold of the sensor. The gas sensor includes a substrate, a first detector disposed on the substrate, electrodes electrically connected to the first detector, and a second detector disposed on the first detector, wherein the second detector has a hydrophilic functional group.

### CROSS REFERENCE TO RELATED APPLICATION

This application claims priority to Korean Patent Application No. 10-2016-0112309, filed Sep. 1, 2016, which is hereby incorporated by reference in its entirety into this application.

### BACKGROUND OF THE INVENTION

### 1. Technical Field

The present invention relates to a highly sensitive carbon-nanomaterial-based gas sensor for use in high-humidity environments and a method of improving the sensitivity thereof. More particularly, the present invention relates to a gas sensor, in which a functional group for binding to a water molecule is formed on the surface of a first detector composed of a carbon nanomaterial, whereby a hydronium ion (H₃O⁺) is produced and thus an additional ion conduction path is formed, thereby obtaining an additional reaction path in high-humidity environments, ultimately improving the sensitivity and detection threshold of the sensor.

### 2. Description of Related Art

Thorough research is ongoing into the use of carbon nanomaterials, including carbon nanotubes, graphene and graphene oxide, in gas sensors due to the excellent electrical conductivity thereof. In particular, graphene is receiving great attention because of its high electromechanical properties based on its two-dimensional structure having an atom-scale thickness.

One-atom-thick single-layer graphene is an ideal material for chemical gas detection because all molecules present on the surface thereof are utilized for gas detection, and moreover, the two-dimensional crystal lattice structure of graphene is able to minimize electrical noise to thus maintain a signal-to-noise ratio at high efficiency in the gas detection.

With regard to graphene, thorough and intensive research is carried out these days in biochemical application fields. For example, the Max Planck Institute in Germany has published study results on graphene-based biosensor platforms that can detect DNA and proteins, and also in Changchun Research Institute, China, DNA-based multiplexer and demultiplexer logic circuits have been studied. In addition thereto, various manufacturing techniques, stacking techniques, and surface functionalization techniques for functionalized graphene are under study.

Among these, graphene surface functionalization technology is capable of diversifying the electrochemical affinity of graphene through the additional electrochemical treatment of graphene or biomolecular binding thereto. This technique may be similarly applied to carbon nanotubes and graphene oxide, as well as graphene.

The functionalized carbon nanomaterial may control the carrier density of the carbon nanomaterial through chemical doping while maintaining the inherent excellent electromechanical properties thereof, thereby actively controlling the early properties of the device. It is expected to be greatly utilized in technical fields where sensitivity and selective sensing capability are regarded as important.

Meanwhile, as the use of gas is increasing day by day in modern society, gas may be helpful for our daily lives, but it may cause serious damage if used incorrectly. Because of this danger, the use of gas sensors is increasing as means for early sensing or detection of combustible or harmful gas in order to preemptively prevent gas damage.

Typically, gas sensors are classified into solid electrolyte sensors, contact combustion sensors, electrochemical sensors, and semiconductor sensors. Among these, semiconductor micro gas sensors are being thoroughly studied these days. This is because a semiconductor micro gas sensor is manufactured or integrated on a silicon chip, thereby exhibiting compatibility with general ICs and low cost and high efficiency of manufacture and use.

The gas sensor is characterized in that the electrical conductivity varies depending on the adsorption of gas molecules, and is based on the principle of measuring the concentration and kind of harmful gas by analyzing changes in electrical conductivity. As described above, the semiconductor gas sensor is mainly used. Recently, a large number of semiconductor gas sensors based on carbon nanomaterials, which are superior in physical and chemical durability and have high electrical conductivity compared to conventional metals, are disclosed.

However, in conventional semiconductor gas sensors, a high operating temperature of 200 to 600° C. or more is required in order to promote the chemical reaction between the sensing film and the atoms, thereby forming an oxygen depletion layer on the surface of the reaction layer. The oxygen depletion layer changes the electron density inside the reaction layer composed of a semiconductor material through electron exchange for specific gases. However, such an oxygen depletion layer binds to water molecules in high-humidity environments, thus decreasing the density of the depletion layer, consequently deteriorating the response of the semiconductor gas sensor, which is undesirable. This problem may also occur in carbon-nanomaterial-based gas sensors.

As such, semiconductor gas sensors for preventing sensitivity from decreasing in high-humidity environments have not yet been developed.

### CITATION LIST

### Non-Patent Literature

(1) K. S. Novoselov, A. K. Geim et al., Nature, 2005, 438, 197-200;

(2) K. S. Kim, Y. Zhao et al., Nature, 2009, 457, 706-710;

### SUMMARY OF THE INVENTION

Accordingly, the present invention has been made keeping in mind the problems encountered in the related art, and the present invention is intended to provide a gas sensor and a method of improving the sensitivity thereof, in which the gas sensor is configured such that a hydronium ion is formed on the surface of a detector through binding to a water molecule in high-humidity environments to thus produce an additional ion conduction path, thereby obtaining an additional reaction path in high-humidity environments, ultimately improving the sensitivity and detection threshold of the sensor.

The present invention is directed to a gas sensor, rather than a biosensor and a humidity sensor, and the gas sensor of the present invention is used to detect liquid or gas VOCs (Volatile Organic Compounds) or other aerobic gases, including, for example, benzene, acetylene, gasoline, paraffin, olefin, and aromatic compounds.

An embodiment of the present invention provides a gas sensor, comprising: a substrate; a first detector disposed on the substrate; electrodes electrically connected to the first detector, and a second detector disposed on the first detector, wherein the second detector has a hydrophilic functional group.

In the embodiment of the invention, the second detector may be configured to form a hydronium ion when reacting with a water molecule.

Furthermore, the second detector may be configured such that an ion conduction path including the hydronium ion is formed on the second detector at a predetermined humidity or more.

In the embodiment of the invention, the second detector may be composed of a material for maintaining a stable stacking structure on the first detector in dry conditions.

Here, the stacking structure of the first detector and the second detector may be formed through π-π stacking.

In the embodiment of the invention, the second detector may comprise a protein.

Particularly, the second detector may be single-stranded DNA.

Here, the functional group may be a hydroxyl group.

Here, the functional group may be a carboxyl group.

In the embodiment of the invention, the conduction path may include the hydronium ion.

In the embodiment of the invention, the first detector may include any one or a mixture of two or more selected from among graphene, graphene oxide, carbon nanotubes (CNTs), nanowire, a photosensitive nanowire film, nanoparticles, and a nano-scale conductive polymer.

In the embodiment of the invention, the gas sensor may further include a cover configured to close the surface of the second detector so as to selectively expose the second detector to air.

Another embodiment of the present invention provides a method of improving the sensitivity of a gas sensor, suitable for gas detection using the gas sensor, which comprises a substrate, a first detector disposed on the substrate, an electrode layer electrically connected to the first detector, and a second detector disposed on the first detector, the method comprising: (a) exposing the second detector having at least one hydrophilic functional group to air under high-humidity conditions of a predetermined humidity or more, (b) reacting the second detector with water vapor for a predetermined period of time, thus forming a conduction path including a hydronium ion, and (c) reacting the gas sensor including the conduction path with a gas to detect the gas, wherein the functional group is a hydroxyl group or a carboxyl group.

In high-humidity environments, the sensitivity of a conventional standard gas sensor is decreased but the gas sensor of the present invention is capable of exhibiting increased sensitivity.

Although techniques for sensing gases to be measured in the state in which the influence of humidity is not sufficiently taken into consideration or humidity is removed are conventionally disclosed, the present invention aims to solve problems related to the humidity in conventional gas sensors and to provide a novel technique therefor.

According to the present invention, the gas sensor can be freely used regardless of weather conditions, humidity conditions in an enclosed space, etc.

### BRIEF DESCRIPTION OF THE DRAWINGS

FIG. 1 shows a gas sensor according to an embodiment of the present invention, including a substrate, a first detector, and electrodes electrically connected to the first detector;

FIG. 2 shows the gas sensor of FIG. 1, further including a second detector;

FIG. 3 shows the production of hydronium when water vapor is adsorbed to the gas sensor of FIG. 2;

FIG. 4 shows the formation of a conduction path based on a proton-hopping mechanism by producing a plurality of hydronium ions;

FIG. 5 shows the principle whereby the sensitivity of the sensor is increased when gas is sensed in the presence of formed hydronium ions according to the embodiment of the present invention;

FIG. 6 shows the binding of water molecules to O, N and H at a predetermined humidity or more in the gas sensor according to the embodiment of the present invention;

FIG. 7 is a graph showing an increase in sensor response due to the hydronium ion channel collapse of the gas sensor according to the embodiment of the present invention;

FIG. 8 shows the gas sensor according to the embodiment of the present invention, further including a cover,

FIG. 9 is a graph showing changes in initial resistance depending on changes in the humidity in the gas sensor according to the embodiment of the present invention;

FIG. 10 is a graph showing the response when gas reacts with graphene and with the second detector of the present invention;

FIG. 11 is a graph showing the response depending on relative humidity;

FIG. 12 is a graph showing the results of testing of long-term stability for the initial resistance of the sensor according to the embodiment of the present invention; and

FIG. 13 is a graph showing the results of testing of long-term stability for the response of the sensor according to the embodiment of the present invention.

FIG. 14 is a graph showing the response of the gas sensor in various humidity environments.

### DESCRIPTION OF SPECIFIC EMBODIMENTS

In the description of the embodiments, it is to be understood that the formation of each layer (film), region, pattern or structure “on” or “under” a substrate, layer (film), region, pad or pattern includes all of the direct formation thereof and formation through an additional layer. The criteria for “top/upper” or “bottom/lower” of each layer are described on the basis of the drawings.

As used herein, the term “connection” includes direct connection and indirect connection of one member to another member, and may represent any physical connection or electrical connection, such as adhesion, attachment, fastening, junction-forming, bonding, adjoining, stacking, etc. and may also include direct connection or indirect connection.

Also, terms such as “first”, “second”, etc. or reference numerals are used only for distinguishing a plurality of elements from one another, and do not limit the order or other features among the elements.

Unless otherwise stated, the singular expression includes a plural expression. In this application, the terms “include” or “have” are used to designate the presence of features, numbers, steps, operations, elements, parts, or combinations thereof described in the specification, and should be understood as not excluding the presence or additional possible presence of one or more different features, numbers, steps, operations, elements, parts, or combinations thereof.

Throughout the drawings, the sizes or shapes of the elements may be exaggeratedly depicted for the sake of clarity of description. Furthermore, terms which are specifically defined taking into consideration the constructions and functions of the present invention are merely set forth to illustrate the present invention, but are not to be construed as limiting the scope thereof.

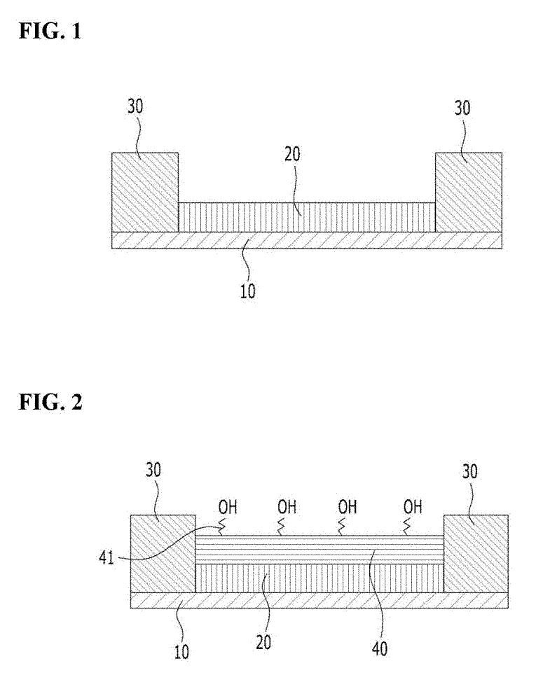

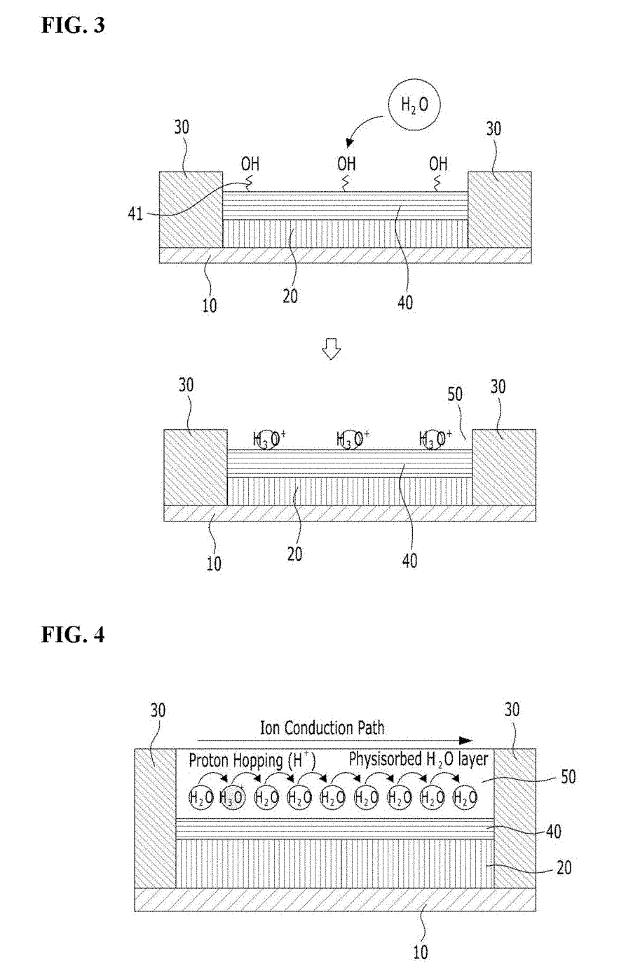

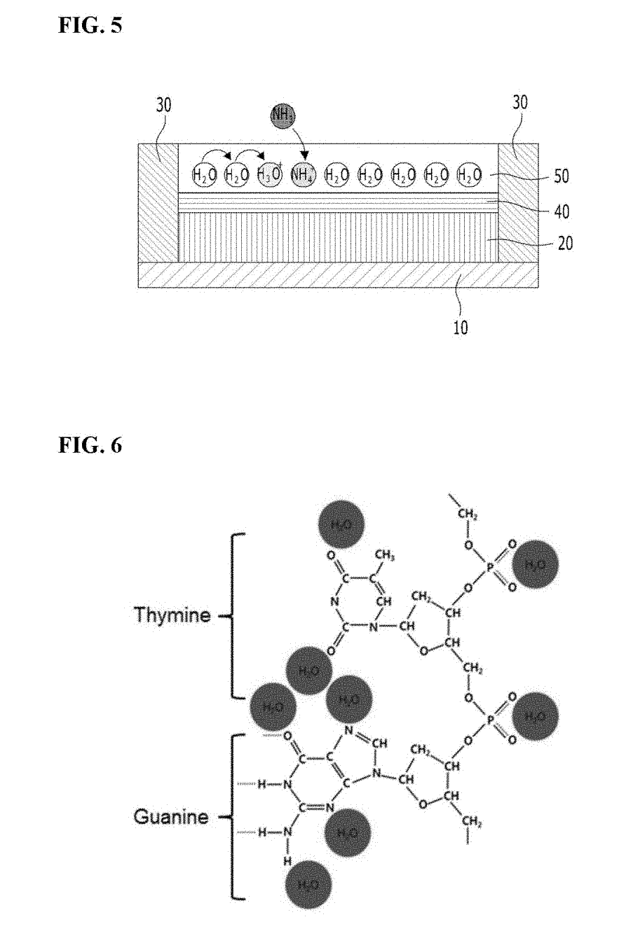

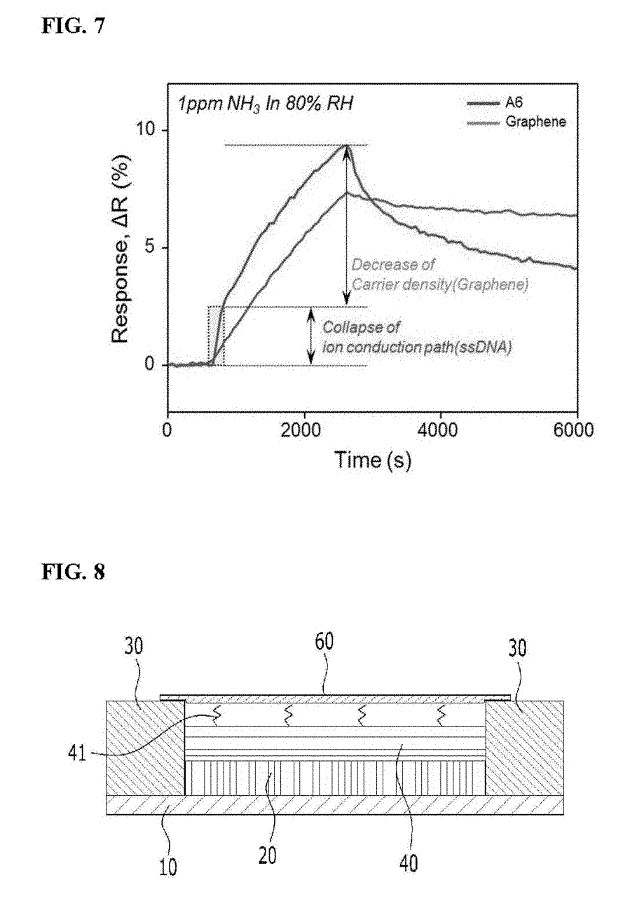

Hereinafter, a gas sensor according to an embodiment of the present invention is described with reference to FIGS. 1 to 8. Here, FIG. 1 shows a gas sensor according to an embodiment of the present invention, including a substrate, a first detector, and electrodes electrically connected to the first detector. FIG. 2 shows the gas sensor of FIG. 1, further including a second detector. FIG. 3 shows the production of hydronium when water vapor reacts with the gas sensor of FIG. 2. FIG. 4 shows the formation of a conduction path based on a proton-hopping mechanism by producing a plurality of hydronium ions. FIG. 5 shows the principle whereby the sensitivity of the sensor is increased when gas is sensed in the presence of formed hydronium ions according to the embodiment of the present invention. FIG. 6 shows the binding of water molecules to O, N and H at a predetermined humidity or more in the gas sensor according to the embodiment of the present invention. FIG. 7 is a graph showing an increase in sensor response due to the hydronium ion channel collapse of the gas sensor according to the embodiment of the present invention. FIG. 8 shows the gas sensor according to the embodiment of the present invention, further including a cover.

According to an embodiment of the present invention, a gas sensor may include a substrate 10, a first detector 20 disposed on the substrate 10, electrodes 30 electrically connected to the first detector 20, and a second detector 40 disposed on the first detector 20.

The second detector 40 may have a hydrophilic functional group. Here, the hydrophilic functional group is a functional group that enables hydrogen bonding with a water molecule at a portion thereof that is exposed to air.

In an embodiment of the invention, the second detector 40 may be configured to form a hydronium ion when reacting with water vapor, and a conduction path 50 including the hydronium ion may be formed on the second detector 40 at a predetermined humidity or more. Here, the conduction path 50 may indicate an ion conduction path, and thus, in high-humidity environments, the sensitivity of the conventional standard gas sensor is decreased, but the gas sensor of the present invention is able to exhibit increased sensitivity.

In the present invention, the substrate 10 may be a printed circuit board (PCB) or a flexible printed circuit board (FPCB). More specifically, the substrate may be rigid or flexible, and may be partially bent, with a curved surface. Thus, the gas sensor of the present invention may be easily attached to the surface of any type of apparatus requiring the gas sensor. In addition to the first detector 20 and the electrodes 30 on the substrate 10, elements such as a driver IC and a communication unit may be disposed. The substrate 10 may be a Si substrate, and in order to increase detection reliability and prevent the generation of noise in a signal, an insulating layer (not shown) comprising SiO₂ may be provided on the substrate 10.

The electrodes 30 may include a plurality of subelectrodes. For example, the formation of the electrodes 30 at opposite ends of the first detector 20 is simply depicted in the drawing, without any direction, but a complicated electrode shape may be applied so long as such electrodes are electrically connected to the first detector 20. Although not shown, the electrodes may be provided in the form of a regular or irregular mesh or in the form in which a (+) terminal and a (−) terminal are disposed in a zigzag arrangement.

Here, the electrodes 30 may include a metal having low resistance, for example, at least one selected from among chromium (Cr), nickel (Ni), copper (Cu), gold (Au), silver (Ag), platinum (Pt), titanium (Ti), aluminum (Al), molybdenum (Mo), palladium (Pd) and alloys thereof.

Meanwhile, in a typical gas sensor, the presence or absence of gas and the response may be checked through changes in resistance depending on the contact reaction between the detector and the gas. Also in the present invention, the gas may be subjected to contact reaction with the gas sensor via the first detector 20, whereby the amount of harmful gas may be measured depending on changes in the electrical conductivity or electrical resistance due to the adsorption of gas molecules. Although the first detector 20 is positioned between the electrodes 30 and has an engraved pattern and is thus disposed lower than the electrodes 30 in the drawing, the present invention is not necessarily limited thereto, and the first detector may be provided in the form of an embossed pattern, that is, the first detector 20 may be disposed higher than the electrodes 30.

In the present invention, the first detector 20 may be composed exclusively of a carbon nanomaterial, or may be configured such that carbon nanotubes are grown on a metal. For example, the carbon nanomaterial of the present invention may include any one or a mixture of two or more selected from among graphene, graphene oxide, carbon nanotubes (CNTs), nanowires, a photosensitive nanowire film, nanoparticles, and a nano-scale conductive polymer.

The carbon nanomaterial has a very large surface-area-to-volume ratio compared to that of a typical metallic detector, and thus exhibits high surface response and is also very efficient in the detection of trace amounts of chemical components. The gas sensor using a carbon nanomaterial, for example, CNTs, is able to measure an electrical signal (conductance, resistance) that is emitted differently depending on the electron properties of gas adsorbed to the nanotubes to thereby sense harmful gas. When CNTs are used for the gas sensor, the operation of the sensor becomes possible at room temperature, and the gas sensor has very good sensitivity due to high electrical conductivity upon reaction with harmful gas such as NH₃, NO₂ or the like, and has high reaction and response rates.

Furthermore, according to the present invention, the first detector (e.g. carbon nanomaterial) may be coupled with the second detector 40, and thus the gas sensor may improve the operating properties in high-humidity environments.

Below, the second detector 40 of the present invention is described in detail.

In an embodiment of the present invention, the second detector 40 may be composed of a material that maintains a stable stacking structure on the first detector 20 in dry conditions. The stable stacking structure may mean that the first detector 20 and the second detector 40 are stacked through π-π bonding. The interconnection between materials is typically carried out using a linker, and the linker is an adhesive material or a specific protein, which is mostly used in an aqueous solution state. The linker is generally present in an aqueous solution state between the materials. However, the gas sensor cannot perform its function in an aqueous solution state, and even when the gas sensor is manufactured so as to enable the sensor function, the application of the linker on the material for detecting gas may decrease the sensitivity of the sensor itself. Conversely, in the present invention, the second detector 40 is formed of a material that is connected to the first detector 20 through π-π stacking, whereby the first detector 20 and the second detector 40 may be connected to each other, even without the use of an additional linker.

Meanwhile, the second detector 40 according to the embodiment of the present invention has a hydrophilic functional group. Here, the term “has” refers to the concept of “includes”, and the second detector 40 is composed exclusively of a material having a hydrophilic functional group, or may be composed not only of the material having a hydrophilic functional group but also of an additional material.

In an embodiment of the present invention, the second detector may be composed of a protein. In the present invention, the protein may include a polypeptide or polypeptides, configured such that many amino acids are connected through peptide bonding, for example, double-stranded DNA and single-stranded DNA, the end of which has a hydrophilic functional group.

Examples of the material for the second detector 40 may include double-stranded DNA and single-stranded DNA, in which a hydroxyl group is formed at the end of DNA comprising combinations of nucleotides A, T, C and G that are interconnected. When this hydroxyl group reacts with water molecules, hydronium ions are produced.

Among double-stranded DNA and single-stranded DNA, single-stranded DNA is preferably used as the second detector 40 of the present invention. Double-stranded DNA is configured such that adjacent bases of different DNA strands are closely connected, and thus the number of hydrogen-bonding sites may be very low. In contrast, in the single-stranded DNA, bases of different DNA strands are not connected to each other, and the number of hydrogen-bonding sites is high, and thus, when the single-stranded DNA comes into contact with water molecules, a large number of hydronium ions may be produced compared to when double-stranded DNA is used.

With reference to FIG. 3, a plurality of single-stranded DNA ends 41 is formed on the second detector 40. When the second detector 40 is composed exclusively of single-stranded DNAs, the single-stranded DNAs may be directly connected to the upper portion (upper surface) of the first detector 20. Here, the term “connection” may mean that the carbon nanomaterial of the first detector 20 and the single-stranded DNA are connected through π-π stacking. The second detector 40 may include single-stranded DNA, and the single-stranded DNA may be one separated from backbones of two strands, the base pairs of which are spirally twisted, based on the double-helix DNA structure.

In the gas sensor of the present invention, when the functional group comes into contact with water vapor (H₂O), a hydrogen bond is formed between the hydrogen atom of the functional group and the oxygen atom of the water molecule. As a result of the above reaction, a hydronium ion (H₃O⁺) is formed, and the hydronium ion (H₃O⁺) is positioned on the top of the second detector (or is directly positioned on the top of the first detector when the second detector is composed exclusively of single-stranded DNA).

As shown in FIG. 4, a kind of ion conduction path 50 may be formed with multiple hydronium ions (H₃O⁺), and the conduction path 50 is responsible for an additional sensing function, as well as the gas sensor measurement function of the first detector 20. That is, the gas sensor of the present invention may be used for detection depending on changes in the resistance of the carbon nanomaterial and on changes in the resistance in the conduction path 50, resulting in high sensitivity.

In the present invention, the second detector 40 does not measure gas all by itself, but measures gas through the conduction path 50 configured to include hydronium ions as a reaction product with water vapor. In high-humidity environments, the top of the second detector 40 is formed with a physisorbed water molecule (H₂O) layer, and the hydrogen atom of the hydronium ion may be coupled with the adjacent water molecule inside the water molecule layer. When the hydrogen atom is coupled with the adjacent water molecule in this way, the ion conduction path is produced through proton hopping.

More specifically, the second detector 40 activates the hydroxyl group (—OH) or carboxyl group (—COOH) on the surface of the gas sensor by virtue of chemical functionalization. For example, when the single-stranded DNA is selected, a solution in which the single-stranded DNA is dissolved in a micromolar amount (e.g. 5 to 25 μmol) is dropped on the surface of the first detector 20 and is then cured in a natural dry state for several hours (e.g. 3 hr), whereby the first detector 20 and the second detector 40 are connected and π-π stacked. The single-stranded DNA end 41 includes a hydroxyl group (—OH), and the hydroxyl group (—OH) is coupled with a water molecule in high-humidity environments to produce the hydronium ion shown in Scheme 1 below.

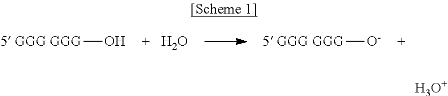

In some cases, the functional group of the second detector 40 may be formed of an inorganic material containing a carboxyl group (—COOH), but in the case of a carboxyl group (—COOH), H⁺ is separated by ether, rather titan typical water vapor, making it difficult to form a hydronium ion (H₃O⁺). Furthermore, the hydroxyl group (—OH) is a functional group that is spontaneously formed at the single-stranded DNA end, whereas the carboxyl group has to be formed so as to react with a water molecule through artificial processing. Thus. the functional group of the present invention is preferably a hydroxyl group (—OH), rather than a carboxyl group (—COOH).

When the first detector 20 reacts with gas, the kind and concentration of gas may be measured through changes in an electrical signal, for example, resistance. Like the first detector 20, the second detector 40 causes changes in resistance when molding with gas. More specifically, when gas is adsorbed to the upper portion of the second detector 40, H⁺ is eliminated from the hydronium ion, and the eliminated H⁺ is linked to the gas molecule, whereby the hydronium ion is reduced to water vapor, which is the original material. For example, as seen in FIG. 5, when the gas molecule, NH₃, is adsorbed on the water layer, hopping of the proton contained in the hydronium is impeded. At this time, since the proton-hopping rate becomes very slow, resistance greatly changes.

After the production of the hydronium ion, the adjacent water molecule may be additionally hydrogen-bonded to the hydrogen portion of the hydronium ion. Furthermore, the hydrogen-bonded water molecule may be continuously hydrogen-bonded to other water molecules, ultimately producing a water molecule (H₂O) layer physically binding to the second detector. The hydrogen atom of the hydronium ion in the produced water molecule layer may freely undergo proton hopping to the adjacent water molecule, thus forming a new ion conduction path through proton hopping.

The above mechanism may be implemented only at a relative humidity equal to or higher than a predetermined value (e.g. 60 to 65% RH). For example, as shown in FIG. 6, the water molecules may be additionally hydrogen-bonded to O, N and H atom portions of the nucleotide and backbone, in addition to the single-stranded DNA end, at a relative humidity of 65% or more. In this way, the additional hydrogen bonding of the water molecules to O, N and H atom portions contributes to the formation of a water molecule (H₂O) layer.

The sensitivity of the gas sensor is determined based on the resistance change (ΔR) upon reaction of the gas and the sensor relative to the initial resistance, and the gas sensor of the present invention is significantly increased in the resistance change relative to the initial resistance by the addition of the basic resistance change of the first detector 20 with the additional resistance change due to the conduction path 50 formed by water vapor in high-humidity environments, compared to typical gas sensors. Such additional changes cause the sensitivity of the gas sensor to increase.

FIG. 7 is a graph showing the resistance change (ΔR) upon reaction of the gas and the sensor relative to the initial resistance over time. For example, when NH₃ gas is dissolved in the water molecule layer, it is coupled with the proton present in the water molecule layer to form an NH₄⁺ ion. In this procedure, the density of protons participating in the ion conduction path is decreased. This means that the ion conduction path breaks due to proton hopping. The collapse of the ion conduction path owing to proton hopping can be confirmed through a drastic increase in the resistance at the early stage of the gas reaction. When comparing the initial response of the sensor (graphene) having no second detector 40 with that of the sensor (A6) including the second detector 40, a drastic increase in resistance can be seen to occur only in the sensor including the second detector 40 at the early reaction.

In the conventional gas sensor, the kind and concentration of gas are measured only through changes in resistance of the first detector 20, and in the present invention, the kind and concentration of gas may be measured through additional changes in resistance by the second detector 40 as well as the first detector 20.

In the conventional gas sensor, techniques for improving sensitivity are disclosed only in a limited manner that amplifies the response for a specific gas, but the present invention is advantageous in that not only maximizing the response for a specific gas but also increasing the total sensitivity of the gas sensor regardless of the kind of gas to be measured may be realized.

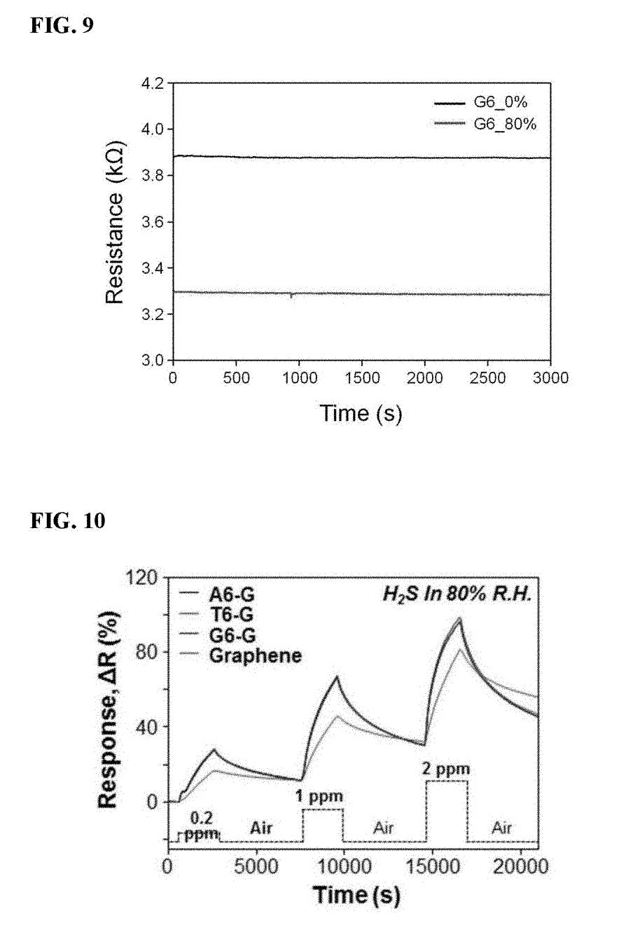

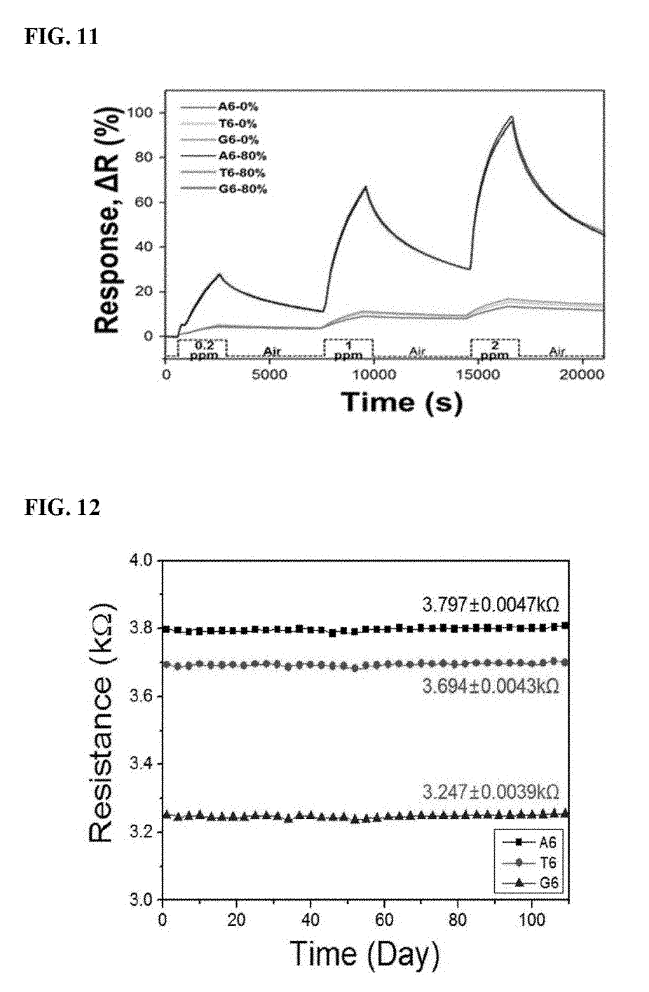

FIG. 9 is a graph showing changes in initial resistance depending on changes in the humidity in the gas sensor according to an embodiment of the present invention. FIG. 10 is a graph showing the response when graphene reacts with the gas and the response when the second detector of the present invention reacts with the gas. FIG. 11 is a graph showing the response depending on the relative humidity.

With reference to FIG. 9, the performance of the gas sensor of the present invention is evaluated on the basis of the initial resistance (kΩ). The initial resistance under high-humidity conditions (a relative humidity of 100%) is decreased compared to the initial resistance under dry conditions (a relative humidity of 0%). Thereby, it can be confirmed that the ion conduction path is formed in high-humidity environments to thus decrease the initial resistance. Based on the test results, the gas sensor of the present invention may exhibit superior performance in high-humidity environments.

With reference to FIGS. 10 and 11, the response varies depending on the gas concentration (0.2 ppm, 1 ppm, 2 ppm), and the response for the specific gas is determined based on the response of the gas measured by the first detector 20, and the second detector 40 functions to improve (amplify) the sensitivity, as shown in the drawing. Amplification of the sensitivity may be achieved through indirect reaction with the gas through the hydronium (H₃O⁺) of the second detector 40.

Meanwhile, the gas sensor of the present invention may further include a cover for closing the surface of the second detector 40 so as to selectively expose the second detector 40 to air. The cover 60 or forming a gas barrier and providing protection may be formed of glass or plastic. In FIG. 8, a cover 60 that closes the opening in the top of the second detector 40 is illustrated. This is to prevent the service life of the gas sensor from decreasing due to the unintentional reaction of the second detector 40 when the gas sensor is positioned under conditions of undesired time and environment, which is merely exemplary and is not necessarily limited to the drawing.

With reference to FIGS. 10 to 13, the method of improving the sensitivity of the gas sensor according to an embodiment of the present invention is described below.

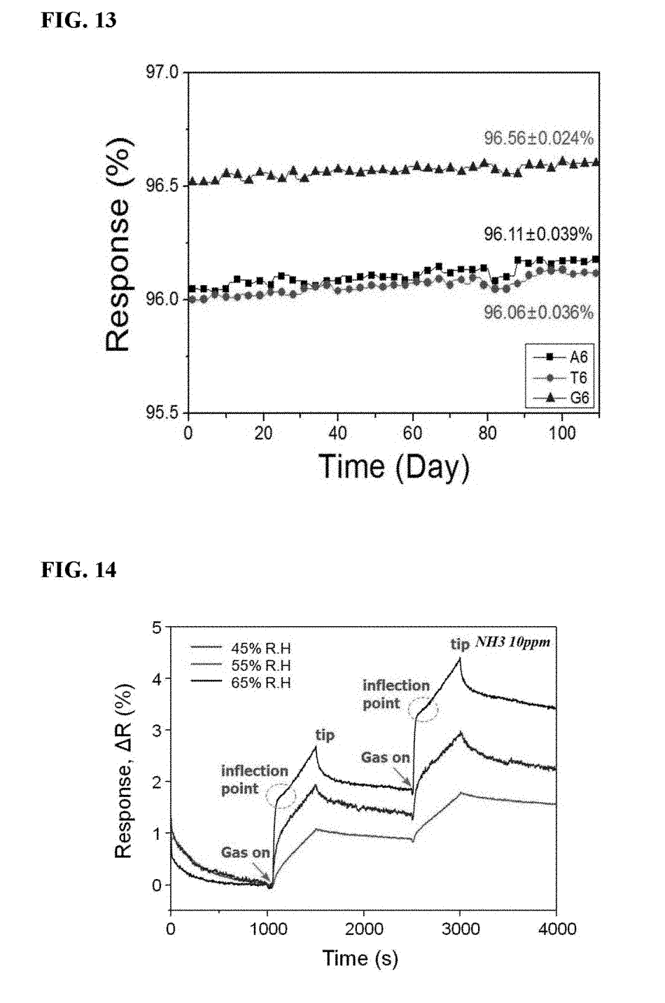

FIG. 12 is a graph showing the results of testing of long-term stability for the initial resistance of the sensor according to an embodiment of the present invention. FIG. 13 is a graph showing the results of testing of long-term stability for the response of the sensor according to an embodiment of the present invention.

The present invention addresses a method of improving the sensitivity of a gas sensor comprising a substrate 10, a first detector 20 disposed on the substrate 10, electrodes 30 electrically connected to the first detector 20, and a second detector 40 disposed on the first detector 20, the method comprising: (a) exposing the second detector 40 having at least one hydrophilic functional group to air under high-humidity conditions of a predetermined humidity or more, (b) reacting the second detector with water vapor for a predetermined period of time, thus forming a conduction path 50 including a hydronium ion, and (c) reacting the gas sensor including the conduction path 50 with a gas to detect the gas.

In order to improve the sensing performance of the gas sensor, the portion of the second detector 40 exposed to air is formed with a functional group, and the functional group, which is a hydroxyl group or a carboxyl group, enables the formation of the conduction path 50 including the hydronium ion through reaction with water vapor, thereby improving the sensitivity of the gas sensor.

In order to evaluate the improvement in sensitivity, the resistance change attributable to the reaction of the water vapor and the hydronium ion may be measured.

As shown in FIGS. 10 and 11, the response is significantly improved according to the above method. Specifically, the second detector 40, including single-stranded DNA composed of A, T and G among nucleotides (A, T, C and G), was used, and the response upon gradual increase in the gas concentration over time was measured.

FIG. 10 is a graph showing the response when the first detector, comprising graphene, reacts with gas and the response when the second detector, formed on the first detector, reacts with gas.

Accordingly, the response can be seen to increase to about 120 to 140% in the predetermined time range when the second detector 40 is formed compared to when only graphene is formed.

Turning to FIG. 11, the response at a humidity of 80% can be found to exceed 200% in the predetermined time range, compared to the response at a humidity of 0%.

The service life of the sensor using an ionic material is remarkably decreased depending on the physicochemical reaction. As seen in FIGS. 12 and 13, whether the sensor may be repeatedly used should be examined.

FIG. 12 is a graph showing the results of testing of long-term stability for the initial resistance of the sensor according to the embodiment of the present invention. FIG. 13 is a graph showing the results of testing of long-term stability for the response of the sensor according to the embodiment of the present invention.

FIG. 12 is a graph showing how stable the gas sensor of the present invention is before gas measurement. As shown in this drawing, the sample of graphene-ssDNA comprising nucleotides A, T and G was observed with an eye to the stability of the initial resistance thereof for 110 days. As results thereof, the stability thereof can be confirmed to be maintained.

FIG. 13 is a graph showing how the response for 2 ppm of hydrogen sulfide (H₂S) gas changes over time at a relative humidity of 100%. Based on the results of observation for 110 days, there were no significant changes.

Even upon continuous and repeated testing for 110 days, long-term stability was maintained, from which the reliability of the gas sensor of the present invention is proven.

FIG. 14 is a graph showing the response of the gas sensor in various humidity environments.

Hereinafter, the humidity may means the relative humidity.

For example, the figure shows the reactivity of the gas sensor in a humidity environment of 45%, a humidity environment of 55%, and a humidity environment of 65%.

Referring to the graph, At the beginning of the gas reaction (“Gas on”), the rate of change in resistance rapidly increases, and after the end of the reaction (“tip”), the rate of change in resistance decreases slowly.

Hereinafter, in the 45% humidity environment and the 55% humidity environment, the reaction rate graph has a monotonous increase shape from “gas on” to “tip”.

However, in a 65% humidity environment, the slope is greater than the 45% humidity environment and the 55% humidity environment.

It is also confirmed that an inflection point is formed in the reaction graph.

In particular, the slope of the graph is formed to be close to infinity (∞) from the point of gas on to the point of inflection.

That is because, in humidity environment of 65%, the hydronium ion and the conductive path including hydronium ion are configured on the second detector.

In condition that the hydronium ion and the conductive path including hydronium ion are configured on the second detector, when the gas reaction occurs, the ion conductive path collapses and then the resistance change ratio is rapidly increases.

High-humidity condition referred to in the present invention can mean an environment in which the hydronium ion and the ion conductive path including the hydronium ion are formed on the second detector.

It has been experimentally confirmed that the ion channel (ion conductive path) is collapsed in a humidity environment of 60% or more and 100% or less (Considering the experimental error), more preferably and more precisely 65% or more and 100% or less.

In addition, a method of manufacturing the gas sensor according to an embodiment of the present invention is briefly described below.

The method of manufacturing the gas sensor according to an embodiment of the present invention includes disposing a first detector 20 on a substrate 10, forming electrodes 30 electrically connected to the first detector 20 on the substrate, and forming single-stranded DNA through spraying or drop coating on the upper surface of the first detector 20 other than the electrodes 30, thus disposing a second detector 40 thereon. Here, the second detector 40 is stacked on the surface of the first detector 20 through π-π orbital bonding, thereby forming the functional group to the end of the portion thereof exposed to air.

More specifically, the electrodes 30 may be formed by depositing a metal material on opposite ends of the detector 20.

The detector 20 may be formed through a photoresist process. The photoresist process is performed in a manner in which photoexposure is conducted through lithography using a mask having the shape of the detector 20 so as to pattern the shape of the detector 20, the patterned photoresist is developed so as to expose the portion of the photoresist other than the shape of the detector 20, and the patterned shape is subjected to oxygen plasma processing to thus etch the shape of the detector 20. When the shape of the detector 20 is exposed by completely removing the photoresist, a carbon nanomaterial such as carbon nanotubes may be directly grown through chemical vapor deposition or a solution including a carbon nanomaterial may be dropped on the surface of the detector 20 so as to generate an electric field. Subsequently, the second detector 40 is applied thinly on the first detector 20 through a spraying process. The second detector 40, subjected to spraying or drop coating, is positioned in the form of a thin film on the first detector 20.

When the gas sensor thus manufactured comes into contact with water vapor in high-humidity environments, hydronium ions are produced, and thus a conduction path is formed on the second detector 40.

Thereafter, when the gas sensor comes into contact with gas, the conduction path gradually breaks, and thus changes in resistance occur more drastically compared to the case of a typical gas sensor. That is, the sensitivity of the gas sensor is remarkably improved.

As described hereinbefore, in high-humidity environments, the sensitivity of a conventional standard gas sensor is decreased, but the gas sensor of the present invention is able to exhibit increased sensitivity. Although techniques for sensing the gas to be measured in the state in which the influence of humidity is not sufficiently taken into consideration or the humidity is removed are conventionally disclosed, the present invention aims to solve problems related to humidity in conventional gas sensors and to provide a novel technique therefor.

The gas sensor is useful in various fields such as industry, agriculture, animal husbandry, office equipment, cooking, ventilation, alcohol testing, air pollution monitoring, combustion control, gas leakage detection, coal oxygen deficiency alarm, fire monitoring, blood gas analysis, and anesthetic gas analysis. In particular, the gas sensor of the present invention can be freely used in the above-mentioned fields regardless of weather conditions, humidity conditions in an enclosed space, and the like. Therefore, the gas sensor of the present invention can be actively employed for products placed in high-humidity environments, such as an air conditioner, a refrigerator, a humidifier, and an air purifier.

The specification is not intended to limit the present invention to the specific terms disclosed. While the present invention has been particularly shown and described with reference to exemplary embodiments thereof, it will be understood by those skilled in the art that various changes, modifications and alterations may be made therein without departing from the scope of the present invention.

The scope of the present invention is defined by the appended claims rather than the foregoing description, and all changes or modifications derived from the meaning and scope of the claims and their equivalents are deemed to be within the scope of the present invention.

What is claimed is:

1. A gas sensor comprising:
a substrate;
a first detector disposed on the substrate;
electrodes electrically connected to the first detector; and
a second detector disposed on the first detector,
wherein the second detector has a hydrophilic functional group.

2. The gas sensor of claim 1, wherein the second detector is configured to form hydronium when reacting with a water molecule.

3. The gas sensor of claim 2, wherein the second detector is configured such that a conduction path including the hydronium is formed on the second detector at a predetermined humidity or more.

4. The gas sensor of claim 1, wherein the second detector is composed of a material for maintaining a stable stacking structure on the first detector in a dry condition.

5. The gas sensor of claim 4, wherein the stacking structure of the first detector and the second detector is formed through π-π stacking.

6. The gas sensor of claim 1, wherein the second detector comprises a protein.

7. The gas sensor of claim 6, wherein the second detector is a single-stranded DNA.

8. The gas sensor of claim 1, wherein the functional group is a hydroxyl group.

9. The gas sensor of claim 1, wherein the functional group is a carboxyl group.

10. The gas sensor of claim 1, wherein the first detector includes any one or a mixture of two or more selected from among graphene, graphene oxide, carbon nanotubes (CNTs), nanowires, a photosensitive nanowire film, nanoparticles, and a nano-scale conductive polymer.

11. The gas sensor of claim 1, further comprising a cover configured to close a surface of the second detector so as to selectively expose the second detector to air.

12. A method of improving sensitivity of a gas sensor suitable for gas detection using the gas sensor comprising a substrate, a first detector disposed on the substrate, electrodes electrically connected to the first detector, and a second detector disposed on the first detector, the method comprising:
a) exposing the second detector having at least one hydrophilic functional group to air under a high-humidity condition of a predetermined humidity or more;
b) reacting the second detector with water vapor for a predetermined period of time, thus forming a conduction path including a hydronium ion; and
c) reacting the gas sensor including the conduction path with a gas to detect the gas.
